(19) United States
(12) Patent Application Publication
(10) Pub. No.: US 2002/0010391 A1
(43) Pub. Date: Jan. 24, 2002

(54) System for administering provings of homeopathic remedies

(76) Inventor: Paul Herscu, Amherst, MA (US)

Correspondence Address: Choate, Hall & Stewart, Exchange Place, 53 State Street, Boston, MA, 02109, US

(21) Appl. No.: 09/906,918

(22) Filed: Jul. 17, 2001

Related U.S. Application Data
(60) Provisional application No. 60/219,295, filed on Jul. 19, 2000.

(51) Int. Cl. A61B 5/00

(52) U.S. Cl. 600/300

(57) ABSTRACT

A computer-based system for conducting homeopathic provings. The provers may be located remotely from the proving administrator, and may enter symptom information via the internet. Provers are preferably supervised by a local homeopath, and enter symptom information categorized by repertory and materia medica chapter. Also included is a system for supplying homeopathic repertory and/or materia medica information wherein users can store and share their own notes and changes to the homeopathic information, and a subscription system for accessing the homeopathic information, which may include clinical data and other supplementary materials.

The present invention claims benefit of priority from U.S. Provisional Application No. 60/219,295, filed Jul. 19, 2001, which is incorporated herein by reference.

### FIELD OF THE INVENTION

The present invention relates to a method of conducting homeopathic provings, and to a system for accessing and updating proving, materia medica, and repertory information for homeopathic medical treatment.

### BACKGROUND OF THE INVENTION

History and Definitions

Homeopathy was developed by Samuel Hahnemann in 1790. While he was translating a materia medica from English into German, he came across a reference that the prevalent prescription for malaria at that time was cinchona bark. The reason stated for its efficacy was that it was quite bitter. Dr. Hahnemann was well versed in the current use of medicine and decided there must be another reason besides its bitter qualities that made it work. He reasoned that other medicines were bitter but were not useful in the treatment of malaria. To prove his point, he experimented on himself by taking cinchona bark and observed the effect. Within a short time he experienced chills, fever, palpitations, sweats, in short, all the symptoms of malaria. He wrote down these effects in the text that he was translating as a footnote.

The more Hahnemann studied and translated medicine and medical texts, the more he observed this phenomenon. If healthy people took massive amounts of a particular drug, the drug would actually cause the same symptoms it was supposed to cure. Hahnemann began to wonder what would happen if you matched the symptoms of a sick patient to symptoms that a drug produced. He began experimenting with this method and developed a new branch of medicine, which he called homeopathy. This is actually the definition of homeopathy, homeo, meaning the same, and pathos, meaning illness. Homeopathic remedies are “potentized” substances: the toxic effects are diminished and the remedial effects increased by a series of dilutions and succusions of the substance.

Hahnemann and some of his healthy colleagues, while in a healthy state, began taking many of these drugs to find their effects on the healthy. They carefully recorded and collated the symptoms that each drug produced. This testing and recording of a homeopathic drug on healthy people is called a proving. Provings are recorded and collated, and then assembled together in a reference text called a materia medica.

The current materia medicas have up to 5,000 proven drugs listed. The drugs are derived from plants, minerals and animal substances. The remedies are listed in alphabetical order in the materia medica. The materia medica has grown to include not just symptoms that were proven but also to include toxicological symptoms as well as symptoms that were actually cured in sick patients using that particular remedy. The symptoms in the materia medica are categorized in order from the top of the body on down. So all the symptoms in the head are categorized together, then the eyes, ears, nose and so on until the extremities.

Some remedies have only 40 symptoms listed in the materia medica, while others have 15,000 symptoms. Since it is impossible to remember all the symptoms of each remedy, about 150 years ago the information was collated into a reference form. All the remedies that affect a certain place in a certain way were placed under a particular category. The book that contained these categories is called a repertory. The categories listed in the repertory are called rubrics.

The repertory of the materia medica is actually a reference tool that lists all the symptoms cured or produced and list every remedy that has treated that particular category/rubric. For example, a rubric might list: Head: pain, above left eye, 3 pm lasting to 6 pm, with one remedy listed under the rubric. Rubrics can be very specific like this one, or very general. A general rubric would be Head; pain, and that general rubric would contain hundreds and hundreds of remedies. The more specific the rubric the better for a homeopath, as it truly indicates a closer match. However, specific rubrics may also be too specific and incomplete and therefore misleading.

Over the years, there have been many changes and additions to the repertories. About fifteen years ago, repertories were computerized into several databases to speed up the search process. About ten ago, an expert system was added to one of the databases to further help with remedy selection by setting certain guidelines that will then give more weight to some rubrics and less weight to other rubrics. Commonly owned U.S. Pat. No. 5,797,839 to Herscu, incorporated by reference herein, describes another computerized system designed to aid the homeopath in choosing the correct remedy for a patient, by analyzing symptoms in terms of a cycle of segments.

Provings

The proving system established by Hahnemann was the first known attempt to isolate the effects of a single drug, during a period when polypharmacy was the norm. Further, the techniques of the double-blind study and placebo testing were introduced into homeopathic medicine in 1885, years before these techniques were adopted by other branches of medicine. All of these techniques are now used in reputable provings.

Homeopathy is based upon the idea that if a substance produces an effect in a large dose, it will tend to cure that effect in a potentized dose. The homeopath attempts to match the symptoms of a patient with the effects of a large dose, and then to administer that remedy in a potentized dose. The better the match between the symptoms produced by the remedy and the symptoms, the greater the likelihood that the remedy is the simillimum that will restore balance and eliminate the symptoms. Thus, homeopathy is dependent on accurate provings data, which provide the necessary symptoms for the remedy so that it can be matched to the symptoms of the patient.

A proving of a homeopathic remedy consists essentially of administering the remedy to a healthy individual and observing the resulting effects. In order to isolate which effects are due to the remedy itself, the prover should be as free of unknown stresses as possible. This means that the prover should be healthy, drug-free, and not experiencing any significant explained or unexplained symptoms that might interfere with the effects of the remedy. Further, a proving should ideally comprise enough different individuals to ensure that symptoms attributable to the remedy can be separated out from random occurrences.

The homeopathic community continually conducts provings of both known and new substances to discover and clarify their effects. A trained homeopath is usually required to sift through the many experiences that a prover experiences when conducting a proving, to find those that are actually attributable to the substance being proved.

However, I have found that it is desirable for this “interpreting” homeopath to be ignorant of the expected characteristics of the substance, so his expectation will not color the results of the proving. The present invention includes the notion that, ideally, a separate homeopath should interpret the symptoms of each prover, so that the observed symptoms of the first prover do not color the interpretation of the symptoms of the second prover. Thus, a proving according to the invention is preferably conducted in a double-blind manner.

The computer-implemented systems of the invention described below alleviate the difficulties associated with supervising a geographically distributed group of provers, where each prover preferably has an associated homeopath aiding in entering the proving symptoms, preferably while maintaining a double-blind study environment so that the results of the proving are not distorted by the expectations of the provers.

### SUMMARY OF THE INVENTION

In one aspect, the invention comprises a system for administering homeopathic provings via a communications network. The system includes a central server (e.g., a personal computer with a Internet connection or a telephone voicemail system), which contains a proving database and a prover database. The prover database contains prover data and passwords for a group of provers, while the proving database contains symptom information for the provers. The server further comprises means whereby a remote prover may enter symptoms experienced during a proving into the proving database. The server may be configured to allow a proving administrator to create a new proving for a particular remedy and potency. If multiple potencies are used (possibly including a placebo), the system may assign different potencies to different patients, without communicating this data to the proving administrator. Provers (or supervising homeopaths) may enter symptoms into the database, which may classify the symptoms by type (e.g., by materia medica/repertory chapter). The server may be further equipped to receive additional symptom information such as a textual symptom description, photos, video, or audio files relating to the symptom, and/or the date, time, and intensity of the symptom. The means for receiving communication from the prover may be an Internet connection.

In another aspect, the invention comprises a system for storing and updating an online repertory, materia medica, or therapeutic. The system comprises a central server with a main database that comprises homeopathic remedy and symptom information, and means for displaying this information at a remote system. The central server is capable of receiving and storing changes to the central database, and of displaying the changed data for the remote user. The change information may be stored separately from the main database, and may be available only to the user submitting the change and/or his designees. The change data may also include data files such as audio or video files, or results from a proving database.

In yet another aspect, the invention comprises a method of supplying up-to-date remedy information to homeopaths. According to this method, a central server comprises a main database of homeopathic symptom and remedy information, and a second database of change information for the main database. The server responds to requests from a remote user by supplying symptom and remedy information from the main database and/or the second database. The user may specify that the information should be that contained in the main database (which typically will correspond to a published repertory), or that the information should come from the main database as modified by the second database. The method may include determining whether the user is entitled to access the change information of the second database before supplying the information. For example, change information may be supplied only to users who subscribe to an updating service. The change data in the second database may include, for example, information from provings not included in the main database, clinical data, or audio or video files.

### BRIEF DESCRIPTION OF THE DRAWINGS

The invention is described with reference to the several figures of the drawing, in which,

FIG. 1 shows a schematic of patient reactions in a proving;

FIG. 2 shows the options available to a proving administrator according to one embodiment of the present invention;

FIG. 3 shows a portion of a symptom report for a proving;

FIG. 4 shows a form for a proving administrator to select symptoms to view;

FIG. 5 is a diagram of a symptom entry screen for a prover;

FIG. 6 shows a form for a proving administrator to unblind a proving; and

FIGS. 7a-7c show sample output for a changeable repertory according to the invention.

### DETAILED DESCRIPTION

Any individual has a propensity or predisposition to experience a variety of interference that in some way stresses him. He responds to the interference in some way. The interference is the “stress” and the response is the “strain.” From this point of view, medicines create unique stresses upon an organism. Additionally, stresses created by medicinal substances generate very specific response patterns from an individual. And these specific responses are generated to better a person's health. This is why we give these medicines. The way this was described before this model was that medicinal substances be defined as those substances that have a positive dynamic action upon the vitality of the organism, most especially the vital force.

From this point of view, potentized substances can now be understood as being a subset of all medicines that provide a rather unique and highly specific stress to the individual. If the potentized substance is “homeopathic” to a sick person, then the straining of the patient, responding to the stress of the potentized substance is so specific as to relieve the person of their chronic complaints.

During a proving, when we give the medicine under experimental terms to a healthy person, the individual is impacted by this unique stress, and will strain in a manner consistent with their unique nature. During a proving one should therefore be able to observe both primary symptoms caused by the stress of the drug upon the organism, and secondary symptoms produced as the individual strains to adapt, evolve, or return to balance.

Primary symptoms in their grossest form can be thought of and seen in the toxicology of the drug. Kent states in his treatise Lectures on Homeopathic Philosophy, “When the patient is under the poisonous influence of a drug it does not seem to flow in the direction of his life action, but when reaction comes then the lingering effects of the drug seems to flow, as it were, in the stream of the vital action. The symptoms that arise are of the best order, and hence it is necessary in proving a drug to take such a portion of the drug only as will disturb and not suspend, as will flow in the stream of the vital order, in the order of the economy, establishing slightly perverted action, and causing symptoms, without suspending action, as we would, for example, with a large dose of Opium.”

When the stress of the drug is great, as in a toxic dose, only a fragment of the true picture of the remedy is seen. These are only the primary symptoms. Homeopaths throughout time have suggested that this type of information is incorrect and not to be relied upon.

Secondary effects are here described as those that are the result of the straining back of the individual toward the healthy state. I, along with Kent, find that these symptoms reflecting this response are of greatest importance in defining the precise nature of a medicine.

One of the benefits of the stress/strain model is that it finally gets rid of the problem of determining which symptoms to use. The topic of primary versus secondary symptoms is one that has dogged homeopathy for 200 years. In many cases we only pay attention to the secondary symptoms. Yet in other situations we pay attention to the primary action.

At the very least the primary action of the stress is showing us the predisposition of the patient to stress and so is showing us qualities of the individual's state. So rather than saying that the primary effects are incorrect, I think a better way to look at them is that they are an incomplete picture of the whole. Just like the secondary symptoms are somewhat incomplete as well. They both reflect one part of the stress and strain, the dance of life, this cycle of existence.

Stress and Strain in Practice and in Provings

In my model, when treating a patient we have emphasized the importance of understanding this process of untuning and retuning. We seek to understand the stresses that have pushed the individual out of the balance of health, and how the individual strains to recover. We see how the individual constantly strives, more or less ineffectively, to throw off the stress. The exact dimensions of how this individual strains defines the clinical state of his pathology. By clearly perceiving the totality of the effort of the individual to return to natural health, to adapt more perfectly to his environment, we will understand the patient better.

It is the same in a proving. By perceiving the totality of symptoms produced solely due to the potentized substance during a proving, we will understand the precise nature of that substance. By carefully documenting the symptoms that arise when a healthy patient takes a substance, what is it that we are doing? We are recording the stress of the substance upon the individual (the primary effect) and even more importantly, recording the symptoms that the individual produces in response to that stress, the strain (the secondary effect).

Stresses Affect Different Individuals Differently

Hahnemann wrote that, “All external forces, and especially medicines, have the propensity to produce specific changes in the health of the living organism in their own characteristic way. But the symptoms proper to a medicine do not all come out in one subject . . . ”

We have defined medicines as substances that create unique strains upon the individual. Additionally, it is apparent from our practice and experience that human beings respond to similar stresses in patterns consistent with their unique constitutional nature. Basically, while the stress is the same, the responses are uniquely the individual's. We could therefore predict that a variety of individuals should produce a variety of responses to the stress of the medicine being proven. Susceptibility or predisposition has a part in it though. When the substance to be tested or proven is tested in a more toxic dose, it will stress the largest number of individuals. But as the substance is increasingly potentized, it becomes a stress to an increasingly smaller number of individuals, just the ones that share the individual predisposition to be affected by a small amount of that substance.

Hahnemann similarly concludes in Aphorism 135, “The total picture of disease symptoms that a medicine can produce approaches completion only after multiple observations have been made on many suitable persons of both sexes, with various constitutions.” And in Aphorism 136, “As we have said, in provings on healthy the changes in health which a medicine can produce cannot all be brought out in any one person, but only in many different people of various physical and psychic constitutions . . . ”

A medicine will theoretically tend to produce all symptoms in all provers, but in reality, when a proving is conducted properly, this does not happen. What we find is that some people develop many symptoms, some develop some symptoms, while others do not develop any symptoms. More often, some of the symptoms that are developed are common to many of the provers, while others are unique to certain individuals.

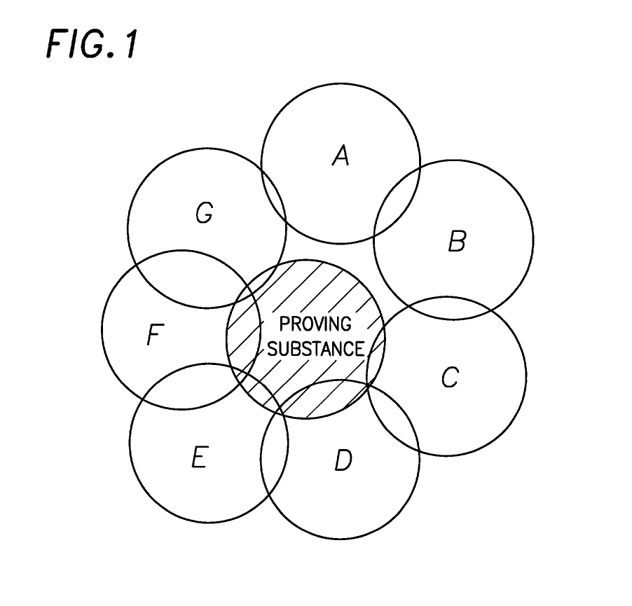

FIG. 1 shows a simplified model of a proving. The central circle represents the symptom picture of the medicine being proved. This circle represents the known or potential symptoms of a proven substance. In either case, think of that inner circle as the sum of the full potential of symptoms that that substance can produce in individuals.

Peripheral circles A-G represent provers with distinct constitutional predisposition and potential symptoms. Again think of these provers as either having symptoms or not, but that the circles represent the full total potential of all the symptoms that that substance can produce in these individuals. As can be seen, not all individuals and their constitutions share the same predisposition. Some provers, such as E and F, have a constitutional predisposition that substantially intersects with the medicine. What this means is that the constitutions of provers E and F both share a fair amount of potential symptoms as the substance.

These symptoms can be seen as “bridges” between remedies. For example, in practice we see a patient remedy state change from one to another; from a constitutional state to an acute state. But if you were to look at the new and the old, you would find that the two states, the two remedies, share a significant bridge between them. Another way of saying this is that any one state predisposes you to enter into another state, but only the ones that share some similar predisposition.

Both constitutions E and F share some similar predisposition with that of the substance to be tested. What we tend to find then is that people of these 2 constitutions are more greatly affected by the substance than other people who do not share these bridges.

FIG. 1 also shows individuals who are not sensitive to the substance being tested. A and B, for example, have little or no intersection with the substance tested. As such, it means that the overall predisposition, the overall sensitivities of individuals in group A and B are substantially different from the substance being proven. As such, this means that we should notice little or no effects when these individuals try to test the substance in the proving, assuming that the stress is not given at a toxic dose.

FIG. 1 also illustrates an observation that was previously unexplainably found in provings. We sometimes find subsets of provers who develop the same symptoms, while others develop completely other symptoms. This model and FIG. 1 conceptualize our findings. Some provers are of the same constitution and therefore develop more similar symptoms. For example, 2 different provers may be in constitution E. As such they share a good deal of predisposition and they respond to stress similarly. When they prove the substance, they develop similar symptoms.

Likewise, provers with different constitutions who have intersections with other provers could share similar symptoms of the remedy, while others may not. For example, in FIG. 1, a prover in group F may develop similar symptoms as a prover in group E because group E and F and the substance have a small space where they intersect. In FIG. 1, it is represented by the intersecting circles of E, F, and the proving inner circle. However, other provers in group F, may still produce symptoms but not be similar to group E. In the Figure, that is demonstrated by the intersection of group F and in the inner circle, but not the part that also intersects with group E.

Still others may develop completely different symptoms. These individuals have a bridge of symptom potential with the inner circle, the substance to be proven, but do not share symptom bridges or potentials with other remedies mentioned thus far. In the diagram group C is such a group. An individual in group C will produce symptoms. Those symptoms may very well be completely different from the symptoms produced by Group E and F as they do not share any symptoms bridges. This is why Hahnemann mentions that we should prove a remedy on many individuals. Conceptually, it means that we are trying to find all the people who have a symptom bridge, have a sensitivity, to the substance. Once we find these people, we should notice that the substance effects them in different ways. The sum total of that experience is what we will call the full potential of the materia medica of the proven substance. Once you removed all other variables, the effect is the effect.

Application

Provings should actually not only be consistent with case taking, but should also give us insight into our clinical philosophy. We have stated that many provers will bring out only certain symptoms of the medicine. The symptoms being manifested are those in common between the nature of the medicine and the nature of that prover's constitutional state. At times, these symptoms become aggravated, and at other times they may even dissipate or resolve, resulting in an overall healthier state for the prover. Hahnemann and others even suggested that because of this observation, properly conducted provings actually strengthen the individual.

Even the best homeopaths will incorrectly prescribe. When a wrong remedy is given, the patient is unwittingly entering a proving. How could one distinguish the two situations? We have shown in our proving model that if the medicine is similar to the constitution of the individual, or if the patient has a high degree of sensitivity, symptoms will be produced. Some symptoms will be the result of the stress of the drug (primary symptoms). “Distinguishing symptoms produced by a simple medicine from those of the disease that it was taken to cure demands the highest discernment . . . Symptoms that were never before noticed, or what were perhaps noticed much earlier in the diseases, are new ones belonging to the medicine”—Hahnemann, aphorism 142.

Some symptoms produced by the patient after an incorrect prescription are actually the result of the patient straining back against the stress of the medicine (secondary symptoms). When medicines are prescribed in potentized form, primary toxic effects are for the most part minimized, while the majority of new symptoms produced will be solely due to the vital response of the patient. These secondary symptoms are common to the given drug and the simillimum of the patient. Those symptoms may resolve, increase in intensity, or lead to temporary or incomplete improvement of the patient.

When a medicine is prescribed that has no apparent effect on the patient, it can generally be assumed that the prescription has little or nothing in common with the necessary remedy. A thorough review of the case and analysis is in order.

Possible Reactions to the Proving Substance

A. Cure of patient. As Hahnemann mentioned “ . . . when the medicine is administered to a person who is sick with similar symptoms, it will exert all its powers, even those that it has seldom revealed in the healthy.” How does this occur? If an individual's constitution coincides fairly exactly with the nature of the medicine being proved, that person will have a dramatic and fairly complete picture of the medicine produced. In this situation, the medicine being proved is considered the simillimum, or at least very similar, to the constitution of the prover, and cure ensues. Many times strange, rare, or peculiar symptoms will be noted in these people, as they are the most sensitive to the substance proven. These symptoms are quite unique to the medicine and have proven quite valuable in the clinical practice of homeopathy.

B. Cure of some symptoms. In some provers, while the person is clearly not cured, they lose some of their chronic symptoms. This occurs because certain segments are shared between the substance being tested and the true simillimum of the prover, so that when the substance stresses the prover, the prover strains back, and strains efficiently enough to cure a symptom. (The division of symptoms into cycles of segments is described in more detail in U.S. Pat. No. 5,797,839 to Herscu, incorporated herein by reference).

C. New symptoms develop. Again, when a substance is taken by a person who shares some of the sensitivities, then the substance stresses the individual. When the substance is in a toxic/strong dose, then most of the provers will develop symptoms, as they are all sensitive, due to our species sensitivities. When the substance is in a minute amount, potentized, then only the extremely sensitive people will develop symptoms. The people who develop them are people who either need this remedy or a remedy similar to it. We know this to be true because they share bridge symptoms. As such, if the substance is not the simillimum, at least it shares segments with the simillimum. We know it was the simillimum only by the fact that the person was cured of their complaints after these symptoms arise.

Another point is that we do not know necessarily if the symptoms are due to the primary or secondary effects of the substance. But nevertheless, from our model, we can say they both “count” for us.

D. Altered symptoms. Again we may notice that at times, a prover's constitutional symptoms are altered in some way. This shows that the prover has a sensitivity to the remedy and that the altered symptoms belong to the new remedy and, in some respect, to the old.

E. Past symptoms return. Here, symptoms that a person had not experienced in along time return. The question has been to which remedy should these count? Actually to both remedies, the one he needs and the one being proven. Why? The person is sensitive enough to the substance to experience the stress and to strain in response. That straining in response produces the symptom. The symptom belongs to both remedies.

F. No new symptoms. Here the symptoms that the person usually has have remained unchanged. In other words, the person did not have any sensitivity to the substance being proven. The symptoms did not change or go away, and the prover did not develop any new symptoms.

To sum up, a proving is therefore a basic scientific experiment of administering minute doses of substances to relatively healthy individuals and recording the changes that result. Medicines can be defined as substances that, in their gross form, have some physiologic, mental, or emotional effect on the organism. Our job in conducting the proving is to accurately record only the effects of this medicine on a variety of individuals. In so doing, a reliable symptom picture of the medicine will be produced. Reliable symptom pictures are essential to effective and reproducible results in prescribing.

Administration of a proving

The present invention includes a method and a computer-based system for conducting a proving. The computer-based system is preferably set up to allow the proving administrator to conduct the proving in a double-blind manner. By employing a client-server architecture with remote access capabilities, provings can be conducted using a geographically diverse group of provers. This feature allows larger provings to be conducted, while still maintaining a requirement that every prover be supervised by a different homeopath, since a large number of homeopaths are not needed in a single geographic region.

The description below describes a preferred embodiment of the invention which is implemented by personal computers connecting to a central server, e.g. via the internet. Those skilled in the art will readily understand that other remote systems may be used to implement the invention, such as voice mail, personal digital assistants, etc.

The necessary steps of designing a proving include selecting a remedy to prove, selecting dosage levels and frequencies, deciding how many provers will receive each dosage (including the placebo), and recruiting, selecting, and instructing the provers (and any supervising homeopaths). Once these steps have been completed, the remedy must be prepared and distributed to the provers, who will take it according to the instructions and record their symptoms for a period of time.

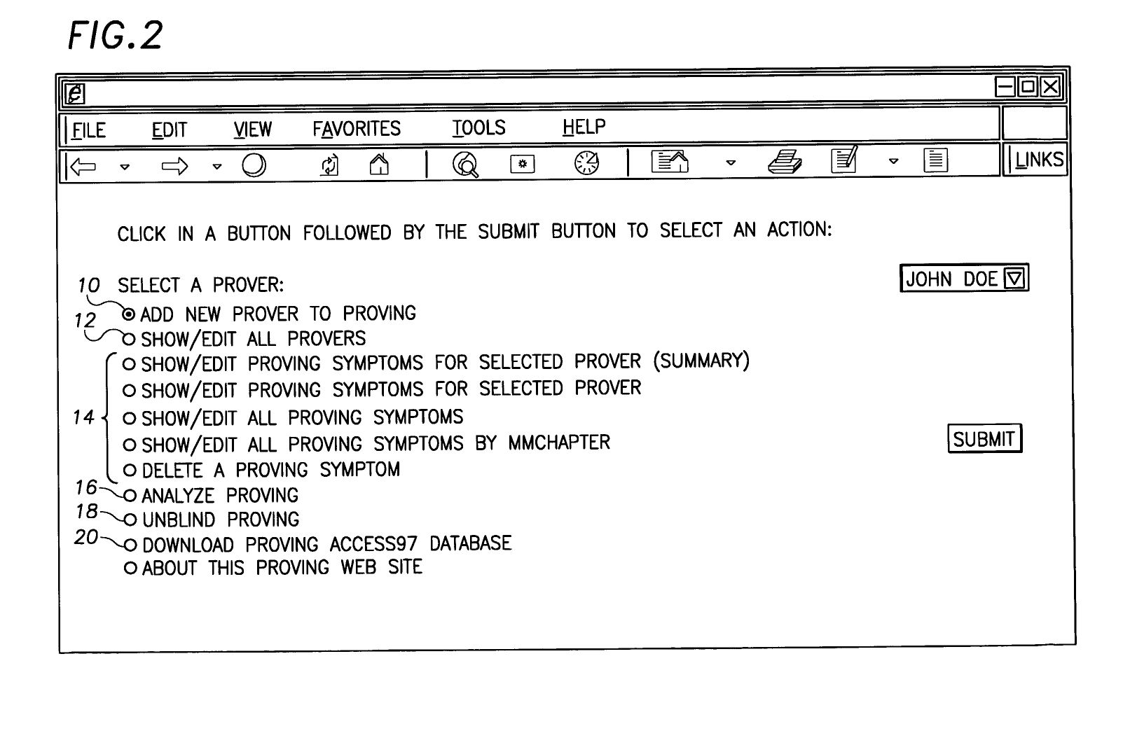

When using the computer-based system of the invention, the proving administrator carries out the above steps (preferably without knowledge of which provers are receiving which potencies of the remedy). Once the provers have been selected, the administrator sets up a new proving on the system by entering their names (or other identifiers) and giving them access to the symptom entry area. A screen capture showing the various functions available to the proving administrator for one embodiment of the invention is shown in FIG. 2.

FIG. 2 shows a set of options available to a proving administrator for editing and viewing a proving. The administrator may add a new prover 10, edit the identifying information for provers already a part of the proving 12, view and edit symptoms entered by the provers 14, view summary information about the types of symptoms experienced by the provers 16, unblind a proving by entering potencies (including placebos) for the different provers 18, or download all of the proving data for use in commercial databases 20.

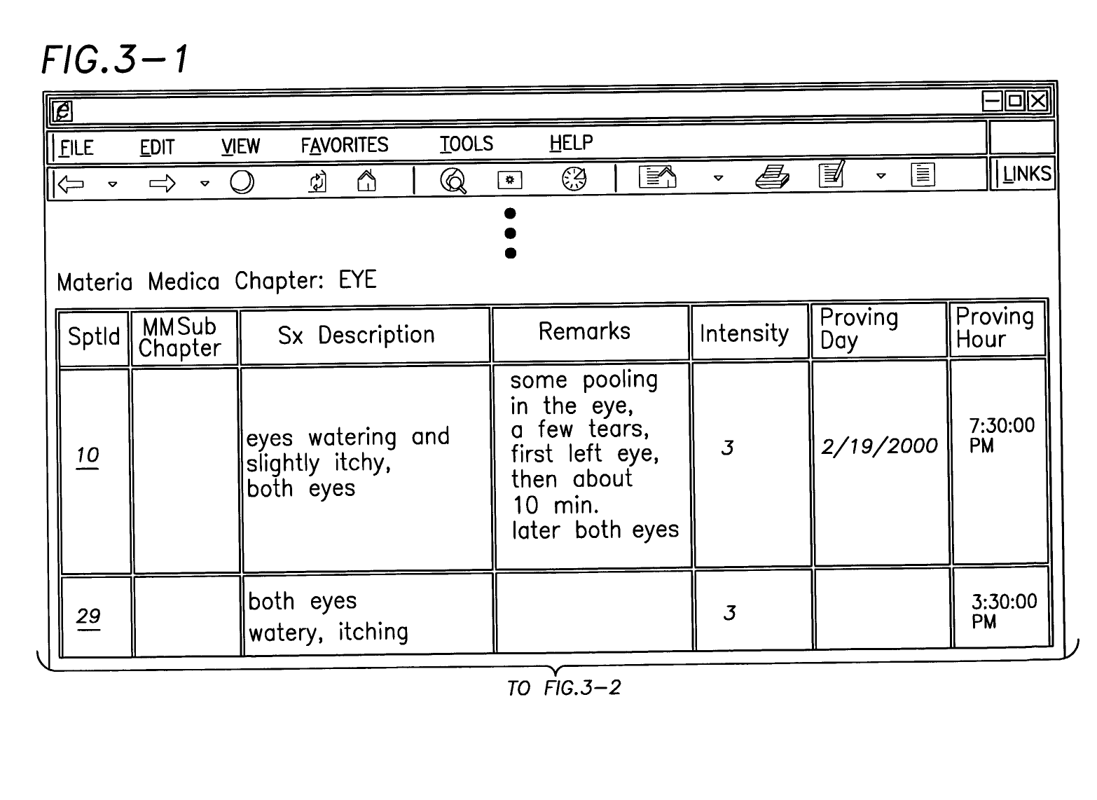

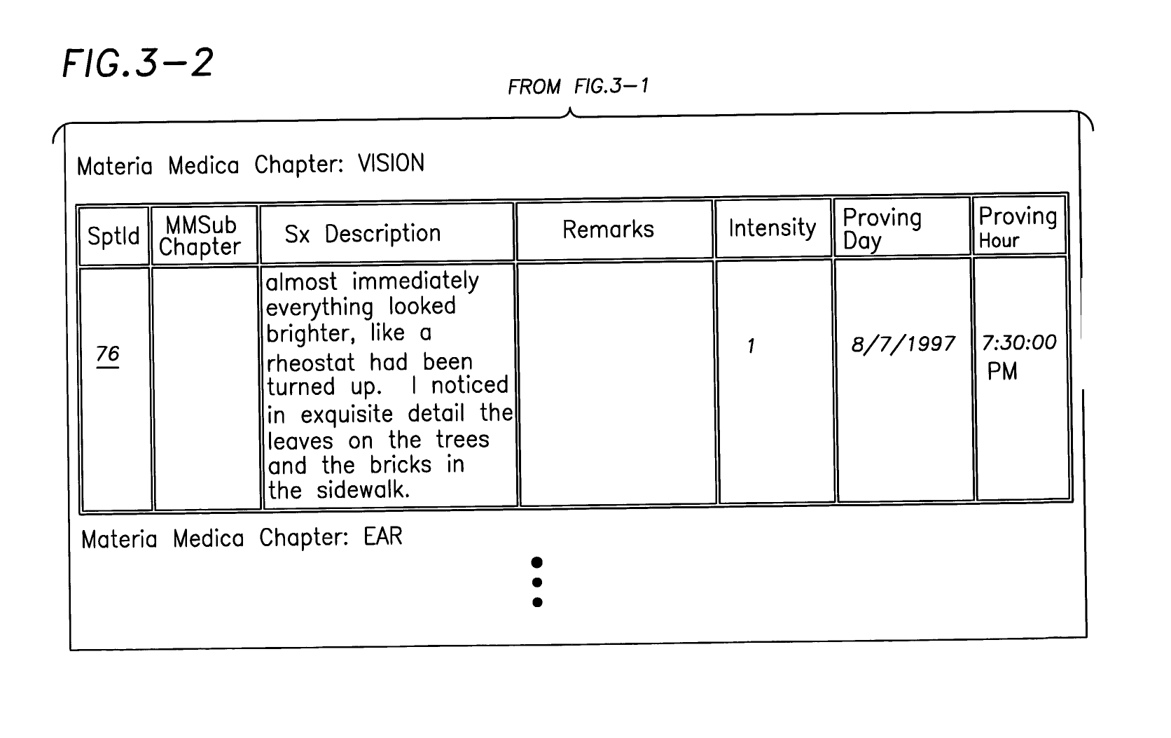

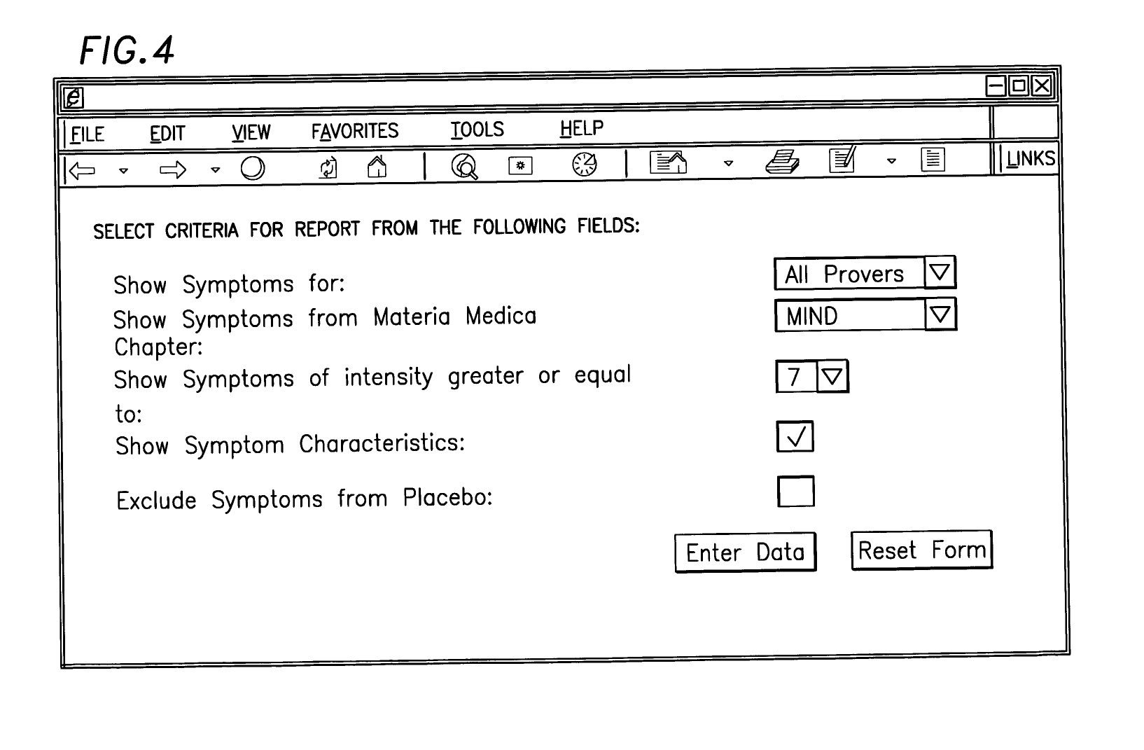

FIG. 3 shows a portion of the information viewable by the proving administrator when “Show/edit all proving symptoms by materia medica chapter” is selected. The figure shows a selection of symptoms relating to the eyes and to vision for different provers. The administrator may choose to view all symptoms, or to view a summary showing only those symptoms of a particular intensity, or (for an unblinded proving) only the symptoms of those provers who received verum. A web form for viewing a summary report in one embodiment of the invention is shown in FIG. 4.

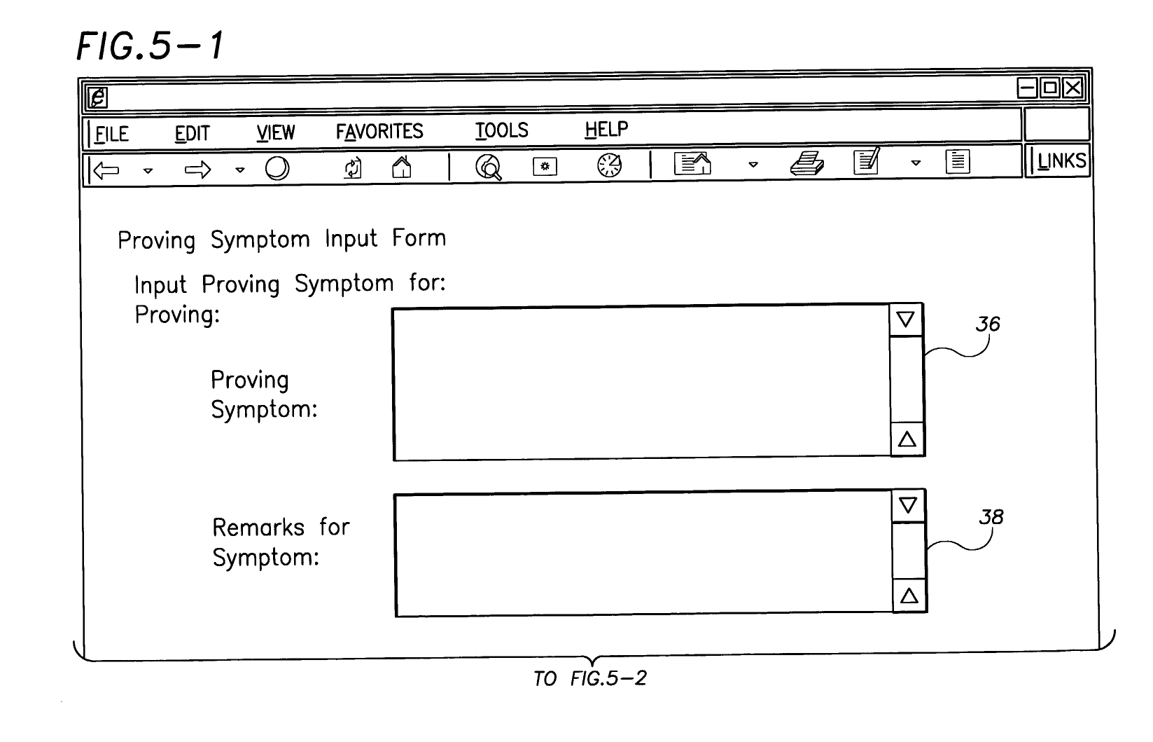

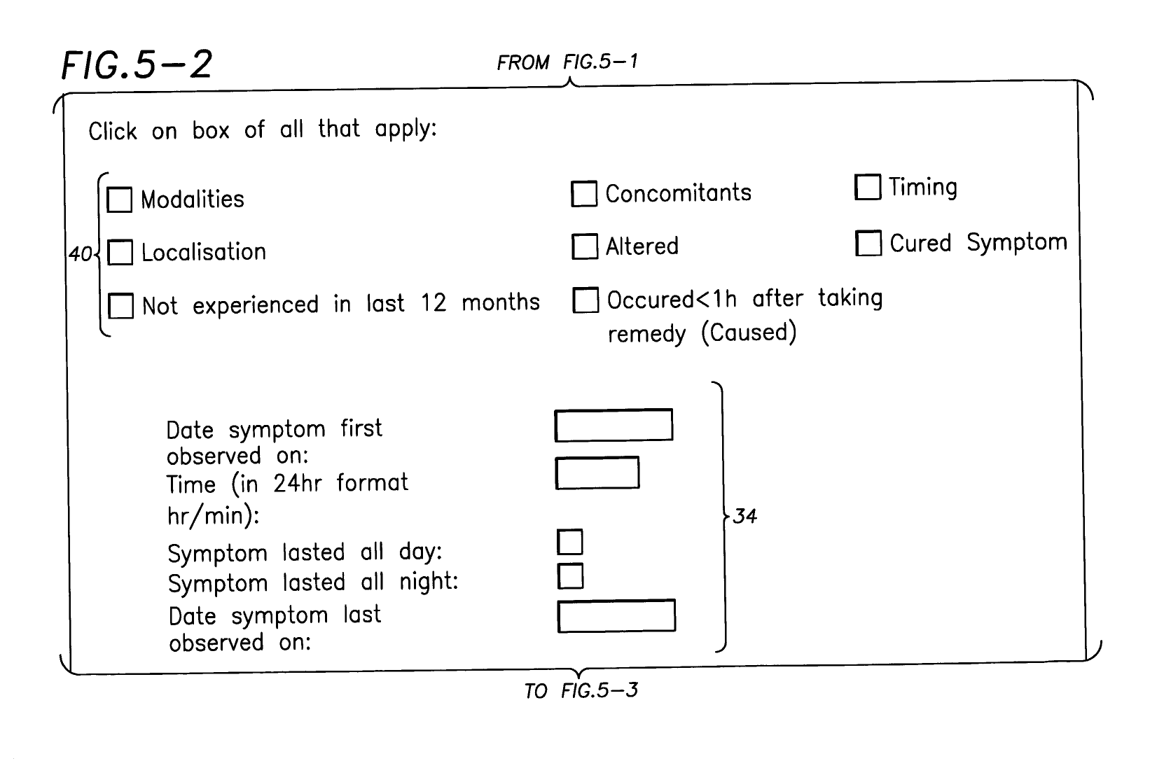

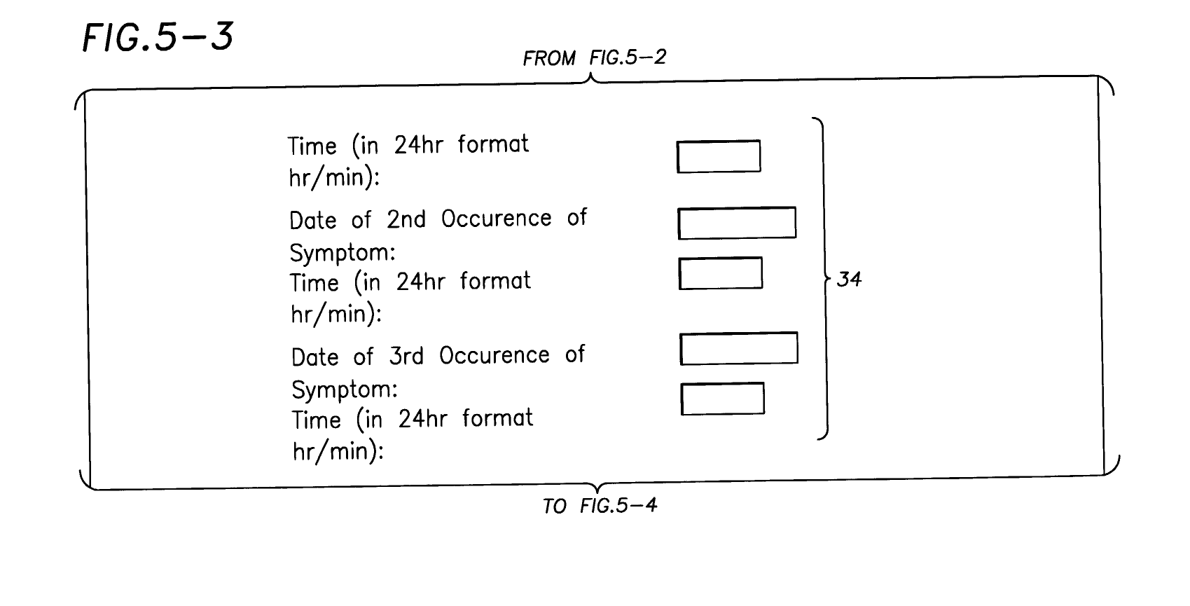

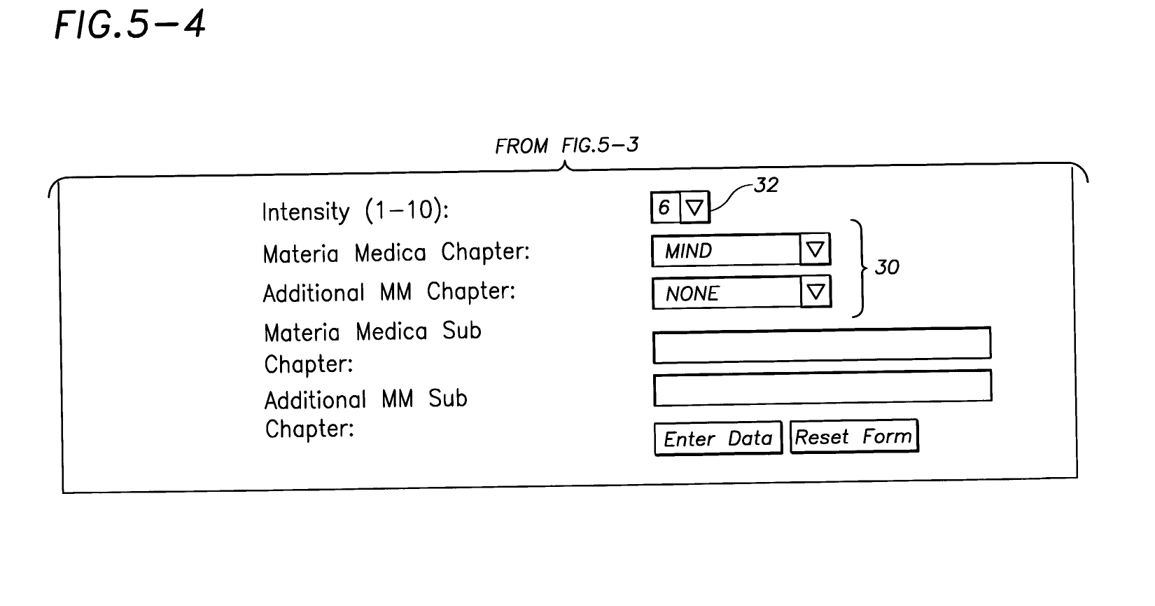

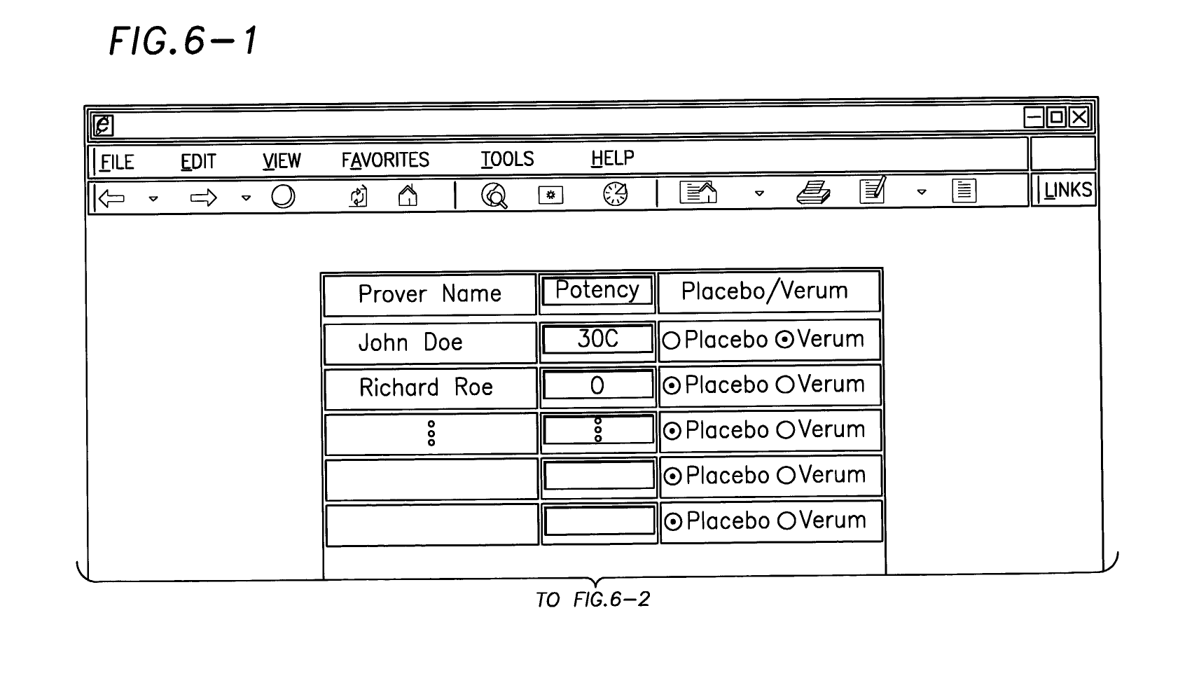

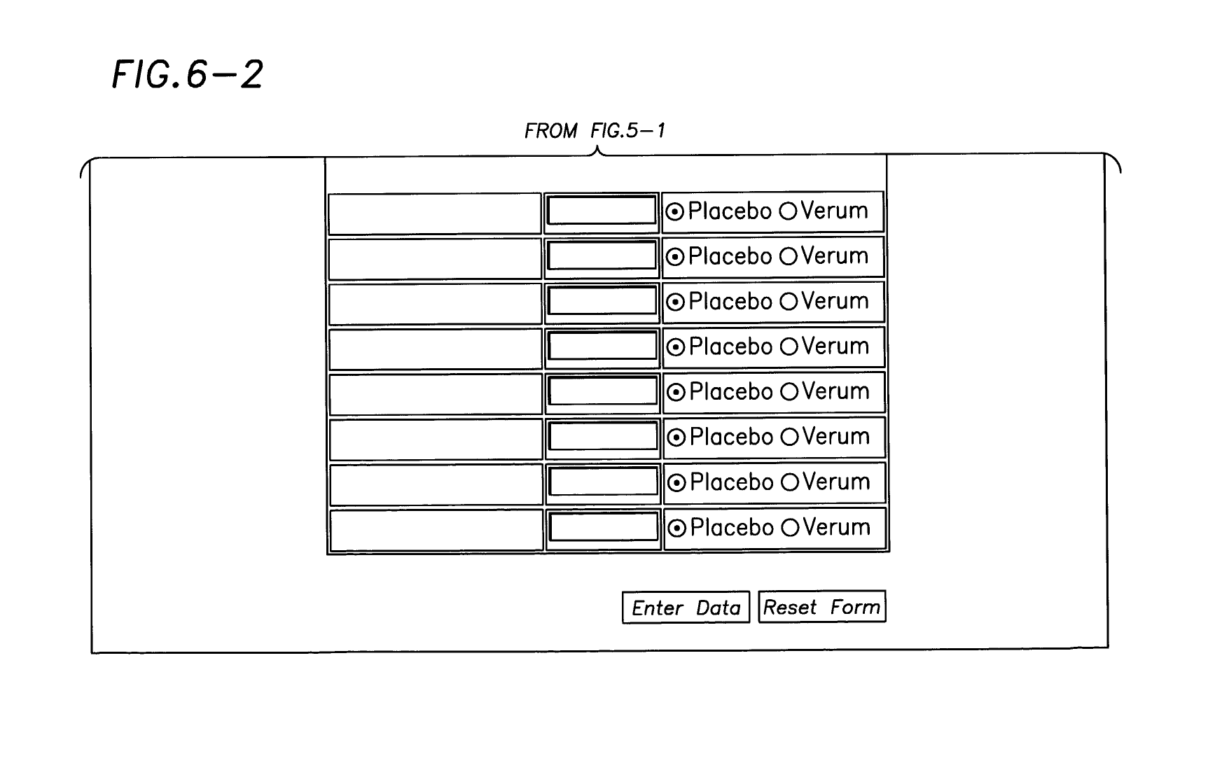

The computer system of the invention provides a convenient way for provers to record their symptoms. Once the proving supervisor has created a logon name and preferably a password for each prover, the user can simply log into the system and enter symptoms. Preferably, the system prompts the user to classify the symptom by materia medica (repertory) chapter, as shown in FIG. 5. The user selects the chapter and optionally the subchapter that corresponds to the symptom 30, a symptom intensity 32, and the date and time that the symptom was experienced 34. He also types in a narrative description of the symptom 36, which will later aid the proving administrator in interpreting the symptom, and may enter remarks 38, for example about the context of the symptom. In the embodiment shown, a set of check boxes 40 is provided for other information about the symptom; the data from these boxes can later be used for viewing and sorting data from the proving. In other embodiments, the patient may be able to upload information such as photos of lesions, or video or audio files relating to symptoms experienced.

In typical use, the patient will confer with a supervising homeopath to determine which symptoms may be attributable to the substance being proved, and these symptoms will be the ones entered into the database. Symptoms may be entered by either the patient or the homeopath. When in doubt as to whether a symptom is attributable to the substance, it is preferable to enter it into the system; the nature of a multipatient proving should prevent random symptoms from being mistakenly associated with the remedy. If there is doubt, the user may note that the symptom is questionable in the remarks section.

Symptom entry into this system is very easy, because the supervising homeopath is very familiar with the organizing principles of the repertory and materia medica, and therefore can accurately classify symptoms in a repeatable way. When necessary, previously entered symptoms can be updated and corrected by the prover. Since the symptoms are already organized by materia medica chapter, the administrator does not need to review and categorize every symptom himself.

Once the proving is completed, the proving administrator can analyze all of the proving data using standard database tools. Laborious data entry is not needed, because the original recording of the symptoms by the user is already stored in digital form on the system. Not only does this speed analysis of the proving, but it prevents transcription mistakes from compromising the data. Analysis may be performed directly on the central server, or the data may be downloaded to a remote computer for viewing by the administrator.

The system is preferably specifically adapted for administration of double-blind studies. Thus, while the proving administrator can view the symptoms as they are being entered, he cannot see which patients have received verum (and at what potency, if multiple potencies are being used) and which have received placebo until the proving is unblinded. In the simplest form of this type of system, the pharmacist maintains a list of which provers received which remedy potencies, and provides the list to the administrator after the proving is over. The administrator can then enter these data manually (“unblinding” the proving) for use in analyzing the proving. FIG. 6 shows a form where the administrator may unblind the proving before analyzing the symptoms. In other embodiments, the program itself maintains the records of potency levels for the different provers. It may, for example, randomly assign potencies according to rules established by the proving administrator, and communicate them directly to the pharmacist for distribution to the provers. In such embodiments, unblinding of the study is automatically done when the administrator indicates that all symptoms have been entered.

Since the proving symptoms are already organized by materia medica chapter in digital form, they can be added to a digital repertory very quickly and easily. In one aspect, the invention comprises a “proving clearinghouse” system, where provings can be conducted online, and their results rapidly incorporated into an ever-changing online repertory. The online repertory is then made available to prescribing homeopaths (e.g. by subscription), so that they always have access to the most up-to-date possible information.

The clearinghouse system is typically stored on a central server. The server may automatically enter the findings of each proving administrator into the online repertory, or it may require that these findings be reviewed for accuracy before entering them into the system. In the latter case, the details of the proving can readily be provided to peer reviewers in a standard format to make the review process as easy as possible.

When desired by the proving administrator, and/or while peer review is pending, the proving administrator may ask that he (and possibly designated others) be able to see the online repertory with his changes included. The changes are stored separately from the “official” version of the repertory, and are dynamically overlaid onto the underlying repertory information shown to the designees of the proving administrator (including himself). For example, the proving may tend to increase or decrease the importance of a particular symptom to a remedy in the repertory, or it may add a new remedy for certain symptoms.

This latter system of separately storing changes to the repertory may also be independently employed. For example, in a system where individual homeopaths subscribe to an online repertory service, they may make their own notations of changes in the repertory. If an individual homeopath feels that the importance of one particular symptom to a particular remedy in the repertory is incorrect, for example, he may “downgrade” that symptom, so it will not be shown to him as having the greater importance when he is searching the repertory. The changes to the repertory associated with each subscriber (and his designees, who may include himself) are separately stored either on the central server or on his own remote system. The changes are then dynamically incorporated into the version of the online repertory that he sees during use, so that it appears to him that the online version fully incorporates his changes and annotations. However, these changes and annotations need not be shown to others unless the homeopath so desires.

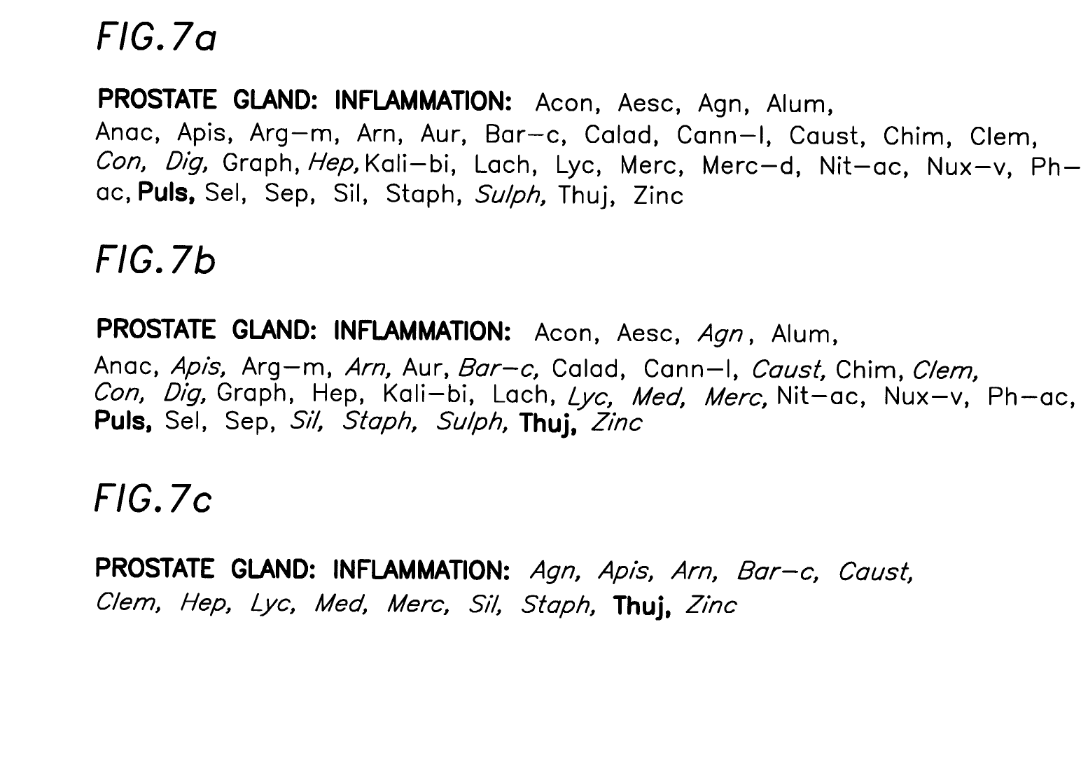

FIGS. 7a-7c schematically show typical outputs for a changeable repertory as described above. FIG. 7a shows a typical remedy list for a particular symptom. This repertory tracks importance of remedies, showing remedies that are particularly likely to be associated with the symptom in italics and remedies that are most likely to be associated with the symptom in bold. FIG. 7b shows a repertory as changed by a particular homeopath. FIG. 7c represents the change information that is stored separately from the main repertory. When the homeopath or his designees accesses the remedy list for the particular symptom, the system dynamically incorporates the information of FIG. 7c with the information of FIG. 7a in order to display the output of FIG. 7b.

The repertory may also be changed, for example, by accessing online information from Centers for Disease Control and/or poisoning hotlines or the like. These data effectively are additional provings data, albeit generally only relevant to relatively toxic remedy potencies. Nevertheless, they can still be used to identify possible new remedies and to add information about the types of symptoms that may be associated with a particular remedy.

The homeopath may also upload other information for storage on the central server such as audio or video, or other data files relating to repertory entries (e.g., video of a patient describing a symptom, audio of a homeopath discussing his prescribing philosophy and clinical experience for a particular remedy, or text files of therapeutics—discussions of groups of remedies). Such material enhances the communication among the homeopathy community in the development of improved treatments. The homeopath may even store his patient files within the repertory system of the invention. He may elect to share symptom and therapeutic response data from his patients, while keeping actual identifying information such as names and insurance numbers private, or he may simply associate patient data with remedies and/or symptoms for his own use.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A system for administering homeopathic provings, comprising:
a central server comprising:
a proving database comprising symptom information for a plurality of provers;
a prover database comprising prover data and passwords for the plurality of provers; and
means for a remote prover to enter symptoms experienced during the course of a homeopathic proving.

2. The system of claim 1, wherein the central server further comprises means for a proving supervisor to create a new proving, by
entering proving data including a remedy, at least one potency level for the remedy, and prover data for one or more provers who will prove the remedy.

3. The system of claim 2, wherein upon creation of a new proving, the central server assigns different potency levels to two provers, without informing the administrator of the potency levels for the provers.

4. The system of claim 3, wherein one of the assigned potency levels is a zero-potency placebo.

5. The system of claim 3, wherein the central server communicates the assigned potency levels to a pharmacist, who provides the potentized remedy to the provers without knowledge of the potency level by the provers or the proving supervisor.

6. The system of claim 1, wherein the symptom entry means comprise means for categorizing symptoms by type.

7. The system of claim 6, wherein the means for categorizing symptoms categorize symptoms according to their chapter in the repertory and materia medica.

8. The system of claim 1, wherein the symptom entry means include means for entering at least one piece of information selected from the group consisting of a narrative symptom description, date, time, intensity, and photo, video, or audio data relating to the symptom.

9. The system of claim 1, wherein the remote prover may communicate symptom information via the Internet.

10. A system for storing and updating an online repertory or materia medica of homeopathic remedies, comprising:
a central server comprising a main database comprising remedy and symptom information identifying symptoms associated with particular remedies;
means for communication with the central database by a remote system, wherein the remote system is adapted to:
display remedy and symptom information from the central database; and
communicate changes in the remedy and symptom information to the central database,
wherein the central database stores the changes in the remedy and symptom information, and upon request from a remote user, displays the changed information to the remote user.

11. The system of claim 10, wherein the changes in the remedy and symptom information are associated with a particular user and stored separately from the main database, and wherein the changed information is viewable only by designees of the particular user.

12. The system of claim 10, wherein the changes in the remedy and symptom information include uploading of data files relating to a remedy or symptom.

13. The system of claim 12, wherein the data files comprise audio or video files.

14. The system of claim 10, wherein the changes in the remedy and symptom information include uploading of an online proving database.

15. A method of supplying up-to-date homeopathic remedy information to homeopaths, comprising:
maintaining a central server comprising
a main database comprising remedy and symptom information identifying symptoms associated with particular remedies; and
a second database comprising changes to the remedy and symptom database; and
responding to requests from a remote user by supplying symptom and remedy information selected from the main database and the second database to the remote user.

16. The method of claim 15, wherein the remote user may specify either that data should be supplied solely from the main database, or that data from the main database should be supplied as modified by data in the second database.

17. The method of claim 15, further comprising determining whether the remote user is designated to receive information from the second database, and either modifying or not modifying data from the main database accordingly.

18. The method of claim 17, further comprising supplying modified data only to users who are subscribers to a repertory updating service.

19. The method of claim 15, wherein the change data of the second database is determined by provings not included in the main database.

20. The method of claim 15, wherein the change data of the second database includes supplementary data selected from the group consisting of clinical data and audio and video data.
